(19) United States
(12) Patent Application Publication
(10) Pub. No.: US 2024/0341757 A1
(43) Pub. Date: Oct. 17, 2024

(54) POWERED SURGICAL DEVICES INCLUDING PREDICTIVE MOTOR CONTROL

(71) Applicant: Covidien LP, Mansfield, MA (US)

(72) Inventors: Christopher Kelly Evans, Southington, CT (US); Thomas Wingardner, North Haven, CT (US)

(21) Appl. No.: 18/658,550

(22) Filed: May 8, 2024

Related U.S. Application Data
(63) Continuation of application No. 17/987,619, filed on Nov. 15, 2022.
Application No. 17/987,619 is a continuation of application No. 16/441,508, filed on Jun. 14, 2019, now Pat. No. 11,497,490.
(60) Provisional application No. 62/695,421, filed on Jul. 9, 2018.

(51) Int. Cl. A61B 17/068; A61B 17/00; A61B 17/072; A61B 17/115; A61B 17/28

(52) U.S. Cl. CPC A61B 17/068; A61B 17/115; A61B 17/282; A61B 2017/00221; A61B 2017/00393; A61B 2017/00398; A61B 2017/00464; A61B 2017/00473; A61B 2017/00486; A61B 2017/00734; A61B 2017/07257; A61B 2017/07271

(57) ABSTRACT

A powered handheld electromechanical surgical device includes a motor configured to drive extension and retraction of a drive component, a sensor configured to sense force exerted on the drive component during extension of the drive component, and a controller including a processor and a non-transitory computer-readable storage medium storing instructions that, when executed by the processor, cause the processor to receive the sensed force from the sensor, control a speed of the motor during extension of the drive component in accordance with the sensed force, determine a speed profile or a force profile during extension of the drive component, and control a speed of the motor during retraction of the drive component in accordance with the speed profile or the force profile.

### CROSS-REFERENCE TO RELATED APPLICATIONS

This application is a continuation application of U.S. patent application Ser. No. 17/987,619 filed on Nov. 15, 2022 which is a continuation of U.S. patent application Ser. No. 16/441,508, filed on Jun. 14, 2019, now U.S. Pat. No. 11,497,490, which claims the benefit of and priority to U.S. Provisional Patent Application Ser. No. 62/695,421, filed on Jul. 9, 2018, the entire contents of each of which being incorporated by reference herein.

### TECHNICAL FIELD

The present disclosure relates generally to surgical devices. More particularly, the present disclosure relates to powered handheld electromechanical surgical devices.

### BACKGROUND

A number of manufacturers have developed surgical devices incorporating powered drive systems for operating and/or manipulating an end effector at a distal end of the device. In many instances, the surgical devices include a powered handle assembly that is reusable and a end effector that is selectively connected to the powered handle assembly prior to use and then disconnected therefrom following use in order to be disposed of or, in some instances, sterilized for re-use.

The use of powered surgical devices such as, for example, electromechanical surgical staplers, has grown tremendously over the past few decades. Advanced technologies and informatics within these intelligent devices provide the ability to gather clinical and operational data that can be used to improve performance, drive design improvements and, ultimately, improve patient outcomes.

### SUMMARY

As detailed herein and shown in the drawing figures, as is traditional when referring to relative positioning on a surgical instrument, the term “proximal” refers to the end of the apparatus or component thereof which is closer to the user and the term “distal” refers to the end of the apparatus or component thereof which is further away from the user. Further, to the extent consistent, any or all of the aspects and features detailed herein may be used in conjunction with any or all of the other aspects and features detailed herein.

Provided in accordance with aspects of the present disclosure is a powered handheld electromechanical surgical device including a motor configured to drive extension and retraction of a drive component, a sensor configured to sense force exerted on the drive component during extension of the drive component, and a controller including a processor and a non-transitory computer-readable storage medium. The storage medium stores instructions that, when executed by the processor, cause the processor to receive the sensed force from the sensor, control a speed of the motor during extension of the drive component in accordance with the sensed force, determine a speed profile and/or a force profile during extension of the drive component, and control a speed of the motor during retraction of the drive component in accordance with the speed profile and/or the force profile.

In an aspect of the present disclosure, the powered handheld electromechanical surgical device according further includes a handle assembly including the motor and controller disposed therein, and an adapter assembly releasably engaged with the handle assembly and including the drive component and sensor disposed therein.

In another aspect of the present disclosure, the powered handheld electromechanical surgical device further includes an end effector releasably engaged with the adapter assembly. In such aspects, extension of the drive component at least one of closes or fires the end effector and retraction of the drive component opens the end effector.

In yet another aspect of the present disclosure, the sensor is a strain gauge.

In still another aspect of the present disclosure, the motor provides a rotational output. The rotational output is converted into translation of the drive component to extend and retract the drive component.

A method of controlling a powered handheld electromechanical surgical device provided in accordance with aspects of the present disclosure includes activating a motor to drive extension of a drive component, sensing force exerted on the drive component during extension of the drive component, controlling a speed of the motor during extension of the drive component in accordance with the sensed force, determining one of speed profile during extension of the drive component or a force profile during extension of the drive component, and controlling a speed of the motor during retraction of the drive component in accordance with the speed profile or the force profile.

### BRIEF DESCRIPTION OF THE DRAWINGS

Aspects and features of the present disclosure are described in detail with reference to the drawing figures wherein like reference numerals identify similar or identical structural elements and:

FIG. 1 is a perspective view of a surgical device configured for use in accordance with the present disclosure and including a handle assembly, an adapter assembly, and an end effector;

FIG. 2 is a perspective view, with parts separated, of the handle assembly of the surgical device of FIG. 1;

FIG. 3 is a perspective view of the adapter assembly of the surgical device of FIG. 1;

FIG. 4 is a perspective view of the end effector of the surgical device of FIG. 1;

FIG. 5A is a graph illustrating exemplary force and motor speed curves during closing and firing of the surgical device of FIG. 1; and

FIG. 5B is a graph illustrating an exemplary motor speed curve during retraction of the surgical device of FIG. 1.

### DETAILED DESCRIPTION

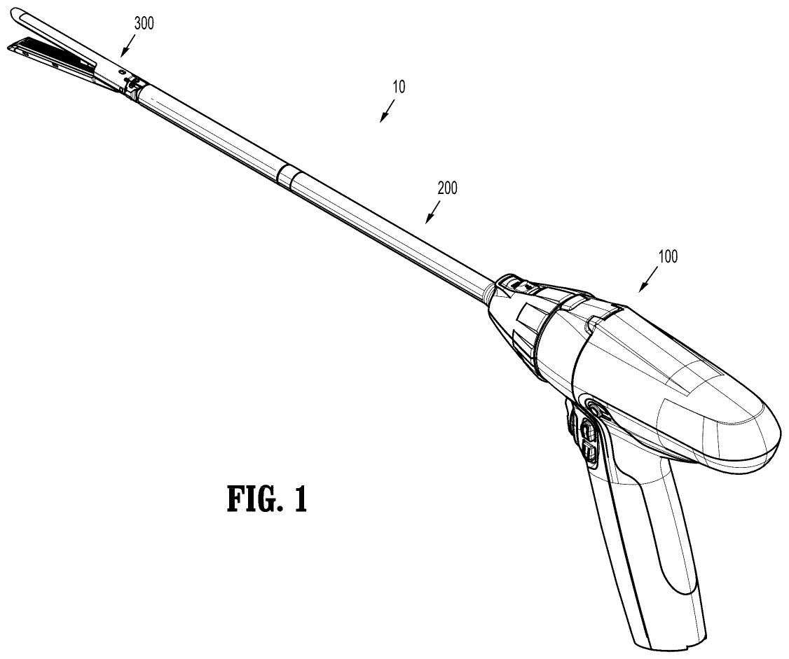

Turning to FIG. 1, a powered handheld electromechanical surgical device exemplifying the aspects and features of the present disclosure is shown generally identified by reference numeral 10. Surgical device 10 includes a handle assembly 100, an adapter assembly 200, and an end effector 300. Handle assembly 100 is configured for selective connection with adapter assembly 200 and, in turn, adapter assembly 200 is configured for selective connection with end effector 300. Although detailed herein with respect to surgical device 10, it is understood that the aspects and features of the present disclosure apply equally to any suitable powered handheld electromechanical surgical device. Thus, surgical device 10 is detailed herein only to the extent necessary to exemplify the aspects and features of the present disclosure. A more detailed description of surgical device 10 can be found in commonly owned U.S. Patent Appl. Pub. No. 2016/0310134, the entire contents of which are hereby incorporated herein by reference.

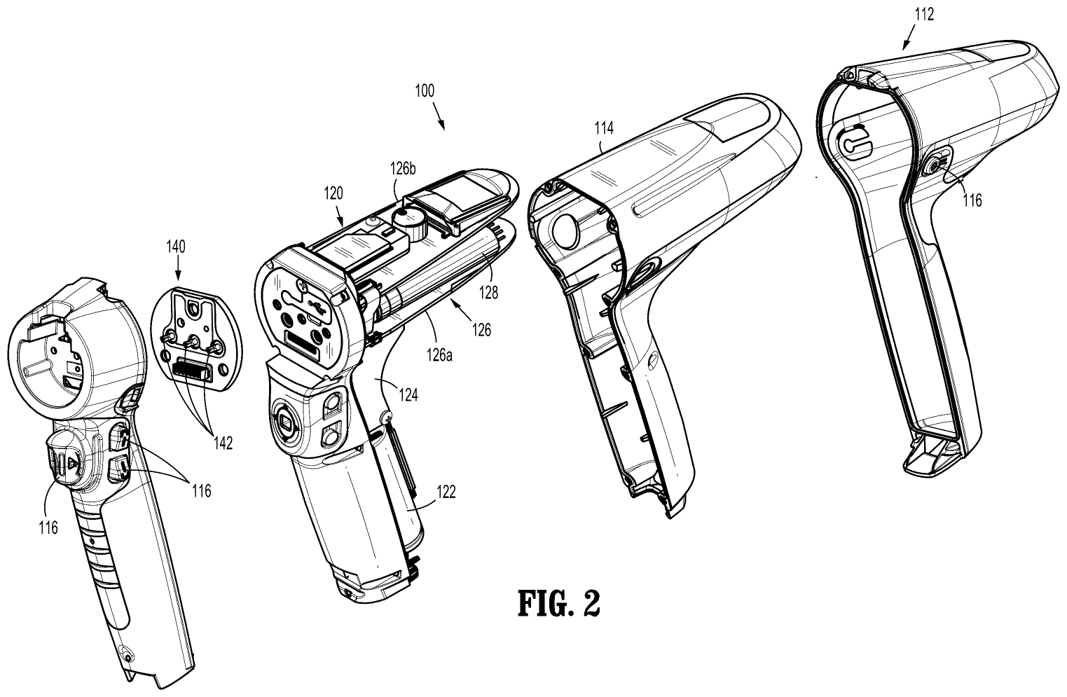

Referring also to FIG. 2 handle assembly 100 generally includes an outer housing shell 112, an inner handle housing 114 disposed within outer housing shell 112, and a power-pack 120 disposed within inner handle housing 114 for powering and controlling the various operations of surgical device 10. A plurality of actuators 116 (e.g., finger-actuated control buttons, knobs, toggles, slides, interfaces, and the like) disposed on outer housing shell 112 communicate with power-pack 120 to enable user-controlled activation of power-pack 120 to perform the various operations of surgical device 10.

Power-pack 120 includes a rechargeable battery 122 configured to supply power to surgical device 10, a battery circuit board 124 (including at least one processor and associated memory), and a controller circuit board 126. Controller circuit board 126 includes a motor controller circuit board 126a (including at least one processor and associated memory) and a main controller circuit board 126b (including at least one processor and associated memory) operably coupled with one another. Motor controller circuit board 126a is operably coupled with battery circuit board 124 enabling communication therebetween and between battery circuit board 124 and main controller circuit board 126b.

Power-pack 120 further includes one or more motors 128 each electrically connected to controller circuit board 126 and battery 122. Each motor 128 includes a respective motor shaft (not shown) extending therefrom for transmitting rotative forces and is controlled by a respective motor controller disposed on motor controller circuit board 126a to enable independent control of each motor 128. Rotation of each motor shaft by its respective motor 128 functions to drive corresponding components of the adapter assembly 200 in order to perform the various operations of surgical device 10, as detailed below. The motor shaft of each motor 128, more specifically, is configured to cooperate with an output shaft 142 of a plate assembly 140 of handle assembly 100 to provide a rotational output from handle assembly 100 to adapter assembly 200.

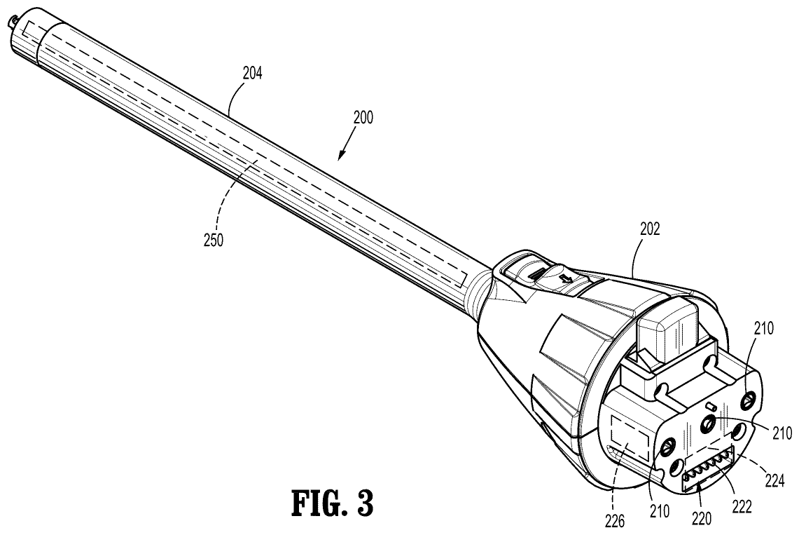

Referring to FIG. 3, in conjunction with FIGS. 1 and 2, adapter assembly 200 includes a connector housing 202 and an outer tube 204 extending distally from connector housing 202. Connector housing 202 is configured for operable connection to handle assembly 100 and the distal end portion of outer tube 204 is configured for operable connection to end effector 300. Adapter assembly 200 further includes one or more rotatable connectors 210 each extending proximally from connector housing 202 and configured to operably couple to a corresponding motor shaft of handle assembly 100 by way of a corresponding output shaft 142 of plate assembly 140 of handle assembly 100 to enable independent rotation of each connector 210 by a respective motor 128, such that rotational force(s) may be selectively transferred from motor(s) 128 of handle assembly 100 to adapter assembly 200. Adapter assembly 200 further includes one or more force/rotation transmitting/converting assemblies (not shown), each extending through connector housing 202 and outer tube 204 and operably coupled to one of the connectors 210. For example, a first force/rotation transmitting/converting assembly may be provided to convert a rotational input from a first of the motors 128 to a first connector 210 into axial translation of an articulation bar (not shown) of adapter assembly 200 to effectuate articulation of end effector 300, a second force/rotation transmitting/converting assembly may be provided to convert a rotational input from a second of the motors 128 to a second connector 210 into rotation of a ring gear (not shown) of adapter assembly 200 to effectuate rotation of adapter assembly 200, and thus, end effector 300, and a third force/rotation transmitting/converting assembly may be provided to convert a rotational input 210 from a third of the motors 128 to a third connector 210 into axial translation of a drive component, e.g., a distal drive member 250 of adapter assembly 200, to effectuate closing, opening, and firing of end effector 300.

An electrical assembly 220 of adapter assembly 200 is supported by connector housing 202 and includes a plurality of electrical contacts 222 extending from a circuit board 224 for electrical connection to handle assembly 100. Electrical assembly 220 also includes a strain gauge 226 electrically connected to circuit board 224, e.g., at least one processor and associated memory thereof, for feedback of closing/firing loads exhibited by adapter assembly 200, e.g., force feedback regarding the distal translation of the distal drive member 250 of adapter assembly 200 to close and fire end effector 300. This force feedback, in turn, is communicated to power-pack 120, e.g., a processor and associated memory of main controller circuit board 126b to, in turn, direct the appropriate motor controller of motor controller circuit board 126a to set the speed current limit on the appropriate motor 128 to ensure closing and firing forces are maintained within acceptable limits. Circuit board 224 further includes a memory configured to store data relating to adapter assembly 200, e.g., identifying information, life-cycle information, system information, force information, which may likewise be communicated to power-pack 120.

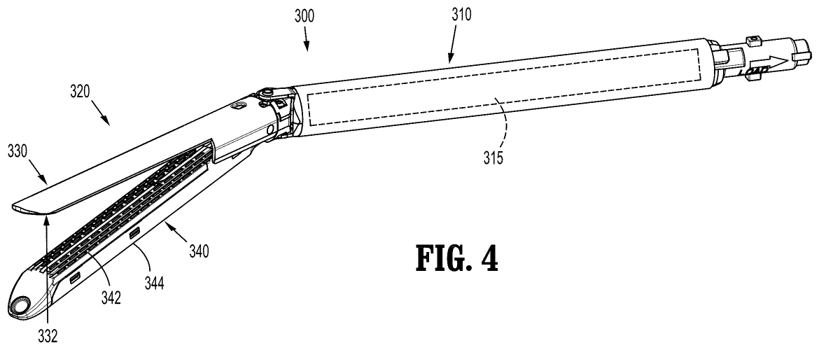

Referring to FIG. 4, end effector 300 is in the form of a linear-stapling, single use loading unit. It should be understood, however, that other types of end effectors may also be used with surgical device 10 of the present disclosure including, for example, end-to-end anastomosis loading units, multi-use loading units, transverse loading units, and curved loading units. The particular end effector 300 utilized with surgical device 10 is recognized by power-pack 120 of handle assembly 100 to enable appropriate operation thereof.

End effector 300 includes a proximal body portion 310 and a tool assembly 320. Proximal body portion 310 is configured to releasably attach to the distal end portion of adapter assembly 200 and tool assembly 320 is pivotally attached to proximal body portion 310. Tool assembly 320 includes an anvil assembly 330 and a cartridge assembly 340. Anvil and cartridge assemblies 330, 340 are pivotal with respect to each other such that tool assembly 320 is movable between an open or unclamped position and a closed or clamped position.

Anvil assembly 330 includes an anvil plate 332 defining a tissue contacting surface (not shown) having a plurality of staple forming pockets (not shown) and a longitudinal slot (not shown) defined therein. Cartridge assembly 340 includes a staple cartridge 342 and a cartridge carrier 344. Staple cartridge 342 defines a tissue contacting surface having staple pockets formed therein for receiving a plurality of staples (not shown) and a longitudinal slot formed in and extending along a substantial length of staple cartridge 342. Cartridge carrier 344 defines an elongated support channel configured to selectively receive staple cartridge 342 therein.

Proximal body portion 310 of end effector 300 includes a drive assembly 315 operably associated with and slidably disposable between anvil and cartridge assemblies 330, 340. Drive assembly 315 includes a drive component, e.g., an elongated drive beam extending to an I-beam including a knife. The I-beam is configured to engage anvil and cartridge assemblies 330, 340 and, upon distal translation relative thereto, pivot anvil and cartridge assemblies 330, 340 relative to one another to close end effector 300 to clamp tissue between the tissue-contacting surfaces of anvil and cartridge assemblies 330, 340. The I-beam is further configured to translate through the longitudinal channels of anvil and cartridge assemblies 330, 340 to drive a sled (not shown) that urges the staples from staple cartridge 342, through clamped tissue, into the staple forming pockets of anvil assembly 330 to fire end effector 300 and form the staples about the clamped tissue. The knife of the I-beam travels through the longitudinal slots defined through anvil and cartridge assemblies 330, 340, to longitudinally cut the clamped and stapled tissue during firing of end effector 300. Drive assembly 315 is operably associated with distal drive member 250 of adapter assembly 200 such that distal translation of distal drive member 250 (effected by a first rotational output received from one of the motors 128 of power-pack 120), is imparted to drive assembly 315 to drive the I-beam to close and fire end effector 300. Proximal translation of distal drive member 250 (effected by a second, opposite rotational output received from one of the motors 128 of power-pack 120), on the other hand, serves to retract drive assembly 315 proximally to return the I-beam to its initial position and open end effector 300 to release the stapled and cut tissue.

For a more detailed discussion of the construction and operation of end effector 300, as illustrated in FIGS. 1 and 4, reference may be made to U.S. Pat. No. 7,819,896, filed on Aug. 31, 2009, entitled “TOOL ASSEMBLY FOR A SURGICAL STAPLING DEVICE,” the entire contents of which being incorporated by reference herein.

Referring generally to FIGS. 1-4, as noted above, distal translation (extension) of distal drive member 250 of adapter assembly 200 is imparted to drive assembly 315 of end effector 300 to close and fire end effector 300. As also noted above, power-pack 120 controls one of the motors 128 to provide a rotational output to adapter assembly 200 that, in turn, is converted (via the corresponding force/rotation transmitting/converting assembly of adapter assembly 200) into distal translation of distal drive member 250 and, thus, drives the closure and firing of end effector 300. More specifically, Power-pack 120, e.g., a processor and associated memory of main controller circuit board 126b, receives force feedback from strain gauge 226 of adapter assembly 200 (via circuit board 224) to set the speed current limit on the corresponding motor 128 to ensure clamping and firing forces are maintained within acceptable limits. Thus, feedback-based control is effectuated whereby the speed of motor 128 may be increased, decreased, or maintained at different points during a clamping and firing operation based upon the clamping and firing forces encountered, as reported by strain gauge 226.

Once firing is completed and it is desired to retract drive assembly 315 and open end effector 300 to release the stapled and cut tissue, power-pack 120 drives one of the motors 128 to provide a rotational output to adapter assembly 200 that, in turn, is converted (via the corresponding force/rotation transmitting/converting assembly of adapter assembly 200) into proximal translation (retraction) of distal drive member 250. This proximal translation of distal drive member 250, as noted above, drives the retraction of drive assembly 315 and opening of end effector 300. However, neither strain gauge 226 nor any other components of end effector 300 or adapter assembly 200 provides force feedback to power-pack 120 during retraction of drive assembly 315 and, thus, feedback-based control as provided during closing and firing of end effector 300 is not available during retraction.

It has been found that controlling retraction of drive assembly 315 is important to manage retraction forces and thereby prevent system damage or malfunction. On the other hand, it is desirable to minimize retraction time to decrease the lengths of surgical procedures and, thus, the time patients are required to remain under anesthesia. However, as noted above, feedback-based control as provided during closing and firing is not available during retraction.

In order to control retraction to manage retraction forces while minimizing retraction time, the force-feedback from strain gauge 226 used to control closing/firing is utilized during retraction, thus obviating the need for retraction force-feedback. More specifically, a memory associated with a processor of main controller circuit board 126b of power-pack 120 (or other suitable memory associated with power-pack 120) is configured to store the force profile (based upon information received from strain gauge 226) and/or motor speed profile (based upon the control of the appropriate motor 128 based upon the force-feedback from strain gauge 226) during closing and firing so that the profile may be utilized to control retraction.

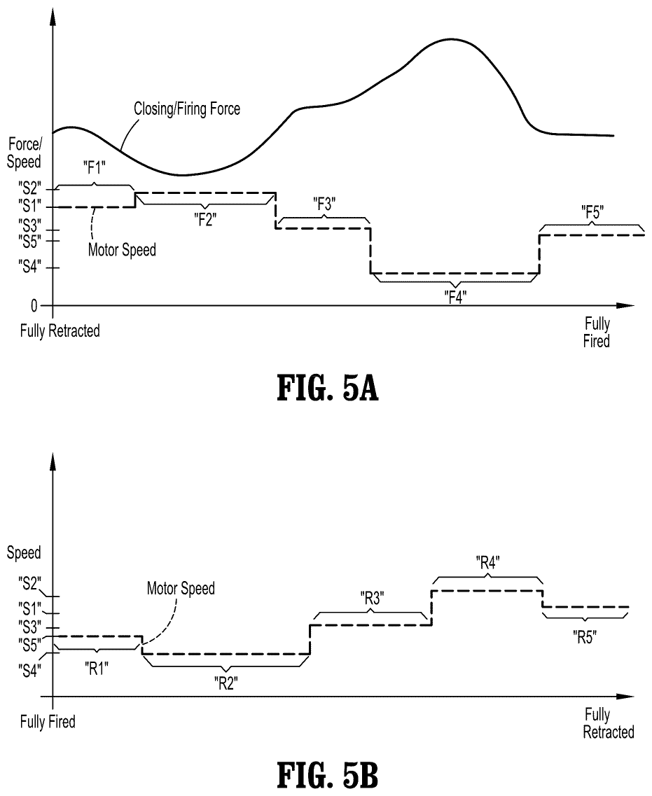

With additional reference to FIG. 5A, sample force and motor speed profiles (for illustrative purposes) for closing/firing are provided. As illustrated, motor 128 is initially driven at a first speed “S1” for an initial, first portion of closing/firing “F1.” In response to decreased forces, the speed of the motor 128 is increased to a second speed “S2” during a second portion of closing/firing “F2.” A subsequent increase in force causes the motor 128 to be decreased from the second speed “S2” to a third speed “S3” during a third portion of closing/firing “F3.” Still further increases in force cause the motor 128 to be decreased to a fourth sped “S4” during a fourth portion “F4” of closing/firing. Finally, a decrease in force causes the motor 128 to increase in speed from the fourth speed “S4” to a fifth speed “S5” during a fifth and final portion of closing/firing “F5.”

Referring also to FIG. 5B, a sample motor speed profile for retraction based upon the sample force and/or motor speed profiles (see FIG. 5A) for closing/firing is provided. As illustrated in FIG. 5B, motor speed is controlled during retraction to account for the portions of retraction where increased forces are likely to be met and/or where decreased motor speeds are likely to be needed, based upon the closing/firing force profile and/or the closing/firing motor speed profile (see FIG. 5A). As understood, retraction is effected in an opposite direction as closing/firing and, thus, the retraction motor speed profile correlates oppositely to the closing/firing motor speed profile. Similarly, the retraction motor speed profile may correlate oppositely with the closing/firing force profile. More specifically, during an initial, first portion “R1” of retraction, which corresponds to the final, fifth portion “F5” of closing/firing, the motor is set to speed “S5.” During a second portion “R2” of retraction, which corresponds to the fourth portion “F4” of closing/firing, the motor is set to speed “S4.” During a third portion “R3” of retraction, corresponding to the third portion “F3” of closing/firing, the motor is set to speed “S3.” During a fourth portion “R4” of retraction, corresponding to the second portion “F2” of closing/firing, the motor is set to speed “S2.” During a final, fifth portion “R5” of retraction, corresponding to the initial, first portion “F1” of closing/firing, the motor is set to speed “S1.” Thus, the motor speed is adjusted to account for the portions of retraction where increased forces may be encountered since increased forces were sensed in the corresponding portions of closing/firing, without the need for force feedback during retraction.

Although illustrated as having the motor speeds directly correspond in FIGS. 5A and 5B, the motor speed of the retraction profile need not correspond 1:1 to the motor speed of the closing/firing profile. For example, a scale factor may be introduced to increase or decrease the motor speed during retraction (or portions thereof) as compared to closing/firing; a dampening or strengthening coefficient may be provided to lessen or exaggerate changes in motor speed during retraction (or portions thereof) as compared to closing/firing; upper and/or lower limits in retraction speed may be imposed regardless of the corresponding motor speed during closing firing; and/or other override rules may be implemented. Additionally or alternatively, the firing speed during closing/firing and/or during retraction may be adjusted between two or more incremental settings (e.g., HIGH and LOW; HIGH, MEDIUM, and LOW; etc.) (which may be the same or different between closing/firing and retraction), or may be adjusted continuously during closing/firing and/or during retraction between upper and lower limits (which may be the same or different between closing/firing and retraction).

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods and techniques that are insubstantially different from those described above and/or in the appended claims are also intended to be within the scope of the disclosure.

1-11. (canceled)

12. A powered electromechanical surgical device, comprising:
a motor configured to drive extension and retraction of a drive component;
a sensor configured to sense force exerted on the drive component during extension of the drive component; and
a controller including a processor and a non-transitory computer-readable storage medium storing instructions that, when executed by the processor, cause the processor to:
receive the sensed force from the sensor;
control a speed of the motor during extension of the drive component in accordance with the sensed force;
generate a speed profile based on the speed of the motor controlled during extension of the drive component from a retracted position to an extended position, wherein the speed profile includes at least one portion corresponding to decelerations in motor speed; and
control a speed of the motor during retraction of the drive component from the extended position to the retracted position to replicate the generated speed profile in reverse.

13. The powered electromechanical surgical device according to claim 12, wherein the sensed force is an initial sensed force, and wherein the controller further causes the process to:
during extension of the drive component, upon receiving a second sensed force from the sensor that is higher than the initial sensed, control the speed of the motor by slowing the speed of the motor from an initial speed of the motor to a second speed of the motor; and
generate the speed profile to include the initial speed of the motor and the second speed of the motor.

14. The powered electromechanical surgical device according to claim 13, wherein the controller further causes the process to:
during retraction of the drive component, following extension of the drive component, control the speed of the motor to replicate the generated speed profile in reverse, such that the motor operates in reverse, first at the second speed of the motor followed by the initial speed of the motor.

15. The powered electromechanical surgical device according to claim 12, wherein the speed profile includes a plurality of portions corresponding to decelerations in motor speed and at least one portion corresponding to an acceleration in motor speed.

16. The powered electromechanical surgical device according to claim 12, further comprising:
a handle assembly including the motor and controller disposed therein; and
an adapter assembly releasably engaged with the handle assembly and including the drive component and sensor disposed therein.

17. The powered electromechanical surgical device according to claim 16, further comprising an end effector releasably engaged with the adapter assembly, wherein extension of the drive component at least one of closes or fires the end effector, and wherein retraction of the drive component opens the end effector.

18. The powered electromechanical surgical device according to claim 12, wherein the sensor is a strain gauge.

19. The powered electromechanical surgical device according to claim 12, wherein the motor provides a rotational output, and wherein the rotational output is converted into translation of the drive component to extend and retract the drive component.

20. A method of controlling a powered electromechanical surgical device, comprising:
activating a motor to drive extension of a drive component;
sensing force exerted on the drive component during extension of the drive component;
controlling a speed of the motor during extension of the drive component in accordance with the sensed force;
generating a speed profile during extension of the drive component from a retracted position to an extended position, wherein the speed profile includes an initial motor speed and at least one portion corresponding to a second motor speed which is different than the initial motor speed; and
controlling a speed of the motor during retraction of the drive component from the extended position to the retracted position to replicate the generated speed profile in reverse.

21. The method according to claim 20, wherein the sensed force is an initial sensed force, and wherein:
during extension of the drive component, upon receiving a second sensed force that is higher than the initial sensed force, controlling the speed of the motor by slowing the speed of the motor from the initial motor speed to the second motor speed; and
generating the speed profile to include the initial motor speed and the second motor speed.

22. The method according to claim 21, wherein:
during retraction of the drive component, following extension of the drive component, controlling the speed of the motor to replicate the generated speed profile in reverse, such that the motor operates in reverse, first at the second motor speed followed by the initial motor speed.

23. The method according to claim 20, wherein the speed profile includes a plurality of portions corresponding to decelerations in motor speed and at least one portion corresponding to an acceleration in motor speed.

24. The method according to claim 20, wherein the speed profile includes a plurality of portions corresponding to progressively stepped decreases in motor speed followed by at least one portion corresponding to a stepped increase in motor speed.

25. The method according to claim 24, wherein:
during retraction of the drive component, following extension of the drive component, controlling the speed of the motor to replicate the generated speed profile in reverse.

26. The method according to claim 25, wherein:
during retraction of the drive component, following extension of the drive component, controlling the speed of the motor to include a plurality of portions of progressively stepped increases in motor speed which correspond to the plurality of portions of progressively stepped decreases in motor speed during extension of the drive component.

27. A method of controlling a powered electromechanical surgical device, comprising:
activating a motor to drive extension of a drive component between a retracted position and an extended position;
during extension of the drive component from the retracted position to the extended position:
sensing a force exerted on the drive component;
controlling a speed of the motor as a function of the sensed force; and
generating a speed profile for the motor, wherein the speed profile includes:
an initial motor speed as a function of an initial sensed force; and
at least one decelerated motor speed as a function of a corresponding at least one increase in sensed force; and
during retraction of the drive component from the extended position to the retracted position:
controlling a retraction speed of the motor, to replicate the generated speed profile in reverse.

28. The method according to claim 27, wherein, during extension of the drive component, upon receiving a second sensed force that is higher than the initial sensed force, the method further comprising:
controlling the speed of the motor by slowing the speed of the motor from the initial speed of the motor to a second speed of the motor; and
generating the speed profile to include the initial speed of the motor and the second speed of the motor.

29. The method according to claim 28, wherein, during retraction of the drive component, following extension of the drive component, the method further comprising:
controlling the speed of the motor to replicate the generated speed profile in reverse, such that the motor operates in reverse, first at the second speed of the motor followed by the initial speed of the motor.

30. The method according to claim 29, wherein the speed profile includes a plurality of portions corresponding to progressively stepped decreases in motor speed followed by at least one portion corresponding to a stepped increase in motor speed.

31. The method according to claim 30, wherein, during retraction of the drive component, following extension of the drive component, the method further comprising:
controlling the speed of the motor to include a plurality of portions of progressively stepped increases in motor speed which correspond to the plurality of portions of progressively stepped decreases in motor speed during extension of the drive component.
